(19) United States
(12) Patent Application Publication
(10) Pub. No.: US 2010/0191735 A1
(43) Pub. Date: Jul. 29, 2010

(54) Systems and methods for classifying and screening biological materials for use for therapeutic and/or research purposes

(75) Inventors: George Reiss, Park City, UT (US); Linda Kelley, Salt Lake City, UT (US)

Correspondence Address: RYAN KROMHOLZ & MANION, S.C., POST OFFICE BOX 26618, MILWAUKEE, WI, 53226, US

(73) Assignee: University of Utah

(21) Appl. No.: 12/653,893

(22) Filed: Dec. 18, 2009

Related U.S. Application Data
(60) Provisional application No. 61/203,107, filed on Dec. 18, 2008.

(51) Int. Cl. G06F 17/30; C12Q 1/68; C12N 5/00; C12N 5/02; C40B 30/02; C40B 50/02

(52) U.S. Cl. 707/740; 435/6; 435/325; 435/375; 506/8; 506/24; 707/802; 707/E17.01; 707/E17.046; 707/E17.002; 707/705

(57) ABSTRACT

Systems and methods collect and screen a biological material to facilitate its selection and use for therapeutic and/or research purposes. The systems and methods take into account both objective and semi-objective attributes manifest in a biological material, such as genetic, phenotypic, clinical, and genealogic attributes. The diverse array of the objective and semi-objective attributes enhances the level of confidence that the biological material can be best matched with a particular research or therapeutic purpose.

### RELATED APPLICATION

This application claims the benefit of U.S. Provisional Patent Application Ser. No. 61/203,107, filed 18 Dec. 2008, and entitled “Systems and Methods for Classifying and Screening Biological Materials for Use for Therapeutic and/or Research Purposes,” which is incorporated herein by reference.

### BACKGROUND OF THE INVENTION

Degeneration of tissues and organs due to aging or injury is the most common cause of morbidity and mortality in society today. New and innovative cellular therapies in the field of regenerative medicine, which use stem and progenitor cells for tissue and organ repair, are being developed throughout the world at an unprecedented rate. Many of these therapies address specific disease states, which are known to have a hereditary disposition or an identified genetic factor. In this field, the source of therapeutic “product” being delivered to the patient will often be obtained from an unrelated human donor. Thus, much of the success of these cellular interventions will rest on the integrity of the donor material or cellular composition.

Inherent to every cell that will be used for therapeutic purposes is a potential to introduce new genetic material to the host, with the possibility of creating a new disease state, perpetuating the current disease state under treatment, or a failure of cellular therapy based on cellular ineffectiveness or lack of potency. With the high cost of developing these therapies and the theoretic risks to the host associated with them, it is prudent to take every measure possible to ensure both the safety and the potency of these cellular products.

Further, the vast majority of basic science research performed in the world today never evolves into a discovery that leads to a cure for disease. This is because the science is limited to the study of single chromosomes, genes, proteins or cells, and in most cases, not of human, origin. In addition, these studies tell us little about disease onset and progression or why some individuals are not susceptible to disease even when certain risk factors are present. Most human diseases likely result from complex mechanisms involving genetic predisposition, environmental factors, previous or coexisting medical conditions, diet, exercise, weight and general overall health. Performing research on stem cells, for example, without knowing what these complex mechanisms are that may have affected the donor organism or their ancestors, and/or without knowing the predisposition of the donor or their ancestors for the presence or absence of a particular disease state, is like trying to fix the lights in a dark room.

### SUMMARY OF THE INVENTION

One aspect of the invention provides systems and methods for collecting and screening a biological material (also called a biosample) to facilitate its selection and use for therapeutic and/or research purposes. The systems and methods include a classifying function that takes into account both objective and semi-objective attributes manifest in a biological material. The objective and semi-objective attributes include genetic, phenotypic, clinical, and genealogic attributes. The diverse array of classification criteria that the classification function considers enhances the level of confidence that the biological material can be best matched with a particular research or therapeutic purpose.

Another aspect of the invention provides a biological material that has been classified according to the classifying function just described.

Another aspect of the invention provides a cataloging function that links an inventory of biological materials to an array of classification criteria, to create a catalog. The classification criteria makes it possible to cross references the biological material in the catalog according to genetic, phenotypic, clinical, and genealogic attributes. The catalog can be embodied in a printed medium or booklet; or be recorded and distributed in a digital medium, such as a CD or DVD; or the contents of the catalog can be incorporated into a relational catalog database program, which dynamically links the inventory of biological materials to the array of classification criteria.

Another aspect of the invention provides an accessing function. The accessing functions couples an interactive interface with a search engine to the catalog database program. The accessing function makes it possible for a user or prospective customer, by use of advanced, multiple-part inquiries or queries entered at a local or remote site, to expeditiously identify and select a biological material from an inventory that is best matched with their particular research or therapeutic objectives.

Another aspect of the invention provides a correlation function that correlates and weighing diverse classification criteria for a given biologiocal material sample, to derive a Biosample Criteria Index (BCI). The BCI can comprise, e.g., a dimensionless, numeric quantity, which expresses the propensity of the donor organism, from which the biological material sample is collected, to manifest a particular selected phenotype and/or genotype. In one embodiment, the classification criteria include data from objective and semi-objective sources, e.g., medical, demographic, epidemiologic, genealogic, and genetic. The BCI further enhances the certainty of the identification and selection process for a biological material for both research and therapeutic indications.

Another aspect of the invention provides a biological material that includes a BCI, as just described.

Another aspect of the invention includes conducting cell therapy and/or tissue engineering using a biologic material classified, at least in part, according to genealogy criteria characterizing lineage information pertaining to a donor of the biologic material, and at least one of a genetic criteria characterizing a genotype of the respective donor and a phenotype criteria characterizing a phenotype of the donor.

Representative embodiments of the invention include the integration of semi-objective genealogic lineage information with other objective, donor-specific classification criteria (such as genetic makeup, genotype, and phenotype). When linked to a biological material sample, the integration of diverse classification criteria can significantly enhance the confidence that a particular biological material can be best matched with a research or therapeutic purpose, can be identified and selected.

The broad-based classification, cataloging, and accessing functions that the invention in its various aspects embodies can be applied to finding a biological material to further research of a particular disease state, i.e., in a research application. For example, when the biological material is sold and distributed for use in a research application, the broad-based classification, cataloging, and accessing functions that the invention embodies make it possible to provide researchers interested in studying a disease state with biosamples known to possess (or not possess) a genetic and/or llineage predisposition for that disease state.

The broad-based classification, cataloging, and accessing functions that the invention embodies can also be applied in finding a biological material to therapeutically treat a human for a particular disease state, i.e., in a clinical application. For example, when the biological material is sold and distributed for use in a clinical application, the broad-based identification and selection process makes it possible to provide clinicians with biologic products with a “cleaner” background or less predisposition to a particular disease state. Biosamples that have been pre-screened for the presence or absence of disease states, using a multitude of medical, demographic, epidemiologic and genetic information, will greatly facilitate discovery and lead to more expeditious cures for these disease states.

### BRIEF DESCRIPTION OF THE DRAWINGS

FIG. 1 is a diagrammatic, pictorial view of a representative system for collecting and classifying biological materials for storage in a repository, to make possible an informed selection by and distribution to members of the health care or clinical research communities for therapeutic and/or, research purposes, the system including (i) a collecting function; (ii) a classifying function; (iii) a cataloging function; and (iv) an accessing function.

FIG. 2 is a diagrammatic, pictorial view of the collecting and entry functions of the system shown in FIG. 1.

FIG. 3 is a diagrammatic, pictorial view of a further aspect of the entry function shown in FIG. 2.

FIG. 4 is a diagrammatic, pictorial view of the classifying function of the system shown in FIG. 1.

FIG. 5 is a diagrammatic, pictorial view of the cataloging function of the system shown in FIG. 1.

FIG. 6 is a diagrammatic, pictorial view of the correlating function that can be part of the cataloging function of the system shown in FIG. 1, in which a BCI is derived.

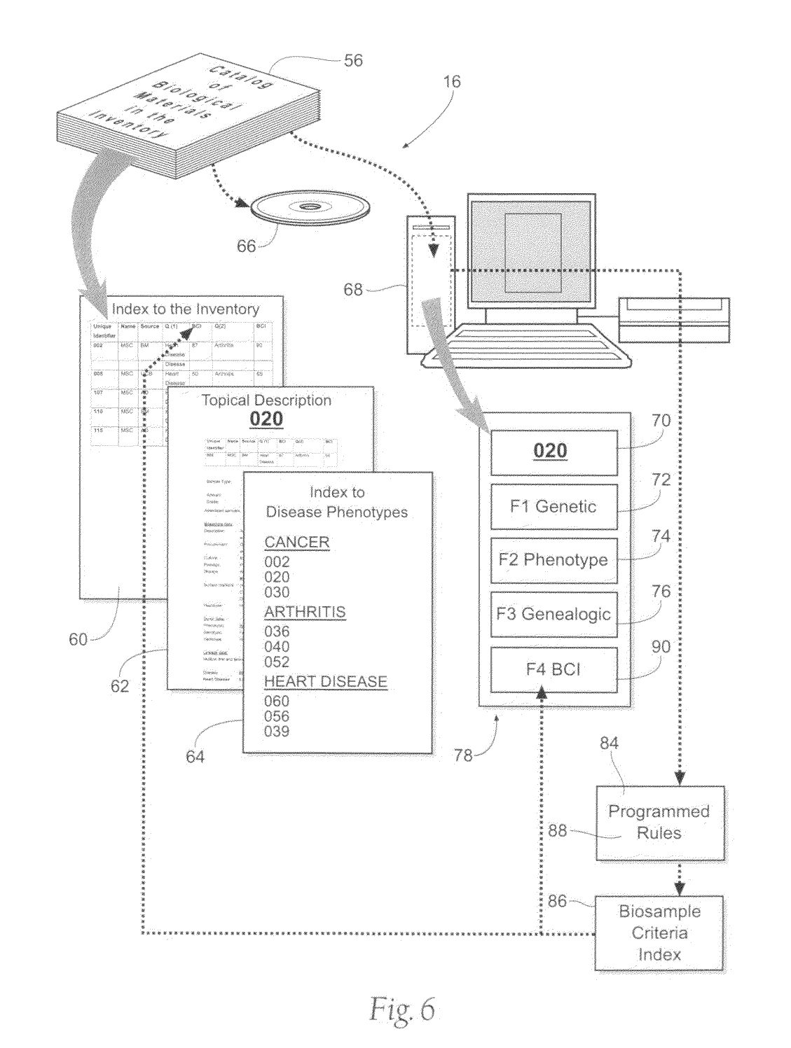

FIG. 7 is a diagrammatic, pictorial view of the accessing function of the system shown in FIG. 1.

### DESCRIPTION OF THE PREFERRED EMBODIMENTS

Although the disclosure hereof is detailed and exact to enable those skilled in the art to practice the invention, the physical embodiments herein disclosed merely exemplify the invention, which may be embodied in other specific structure. While the preferred embodiment has been described, the details may be changed without departing from the invention, which is defined by the claims.

### Contents

The description that follows is divided into the following main sections and sub-sections:

### I. Overview of the System

A. The Collecting Function

B. The Classifying Function
1. Unique Identifier
2. Classification Criteria
   i. Genetic Criteria Component
   ii. Phenotype Criteria Component
   iii. Genealogy Criteria Component
3. Incorporation of Classified Biosamples with Cell Therapy and/or Tissue Engineering

C. The Cataloging Function
1. The Catalog
2. The Biosample Criteria Index

D. The Accessing Function Example

E. Integration of Genealogy Criteria With Routine Patient Monitoring/Diagnosis

### I. Overview of the System

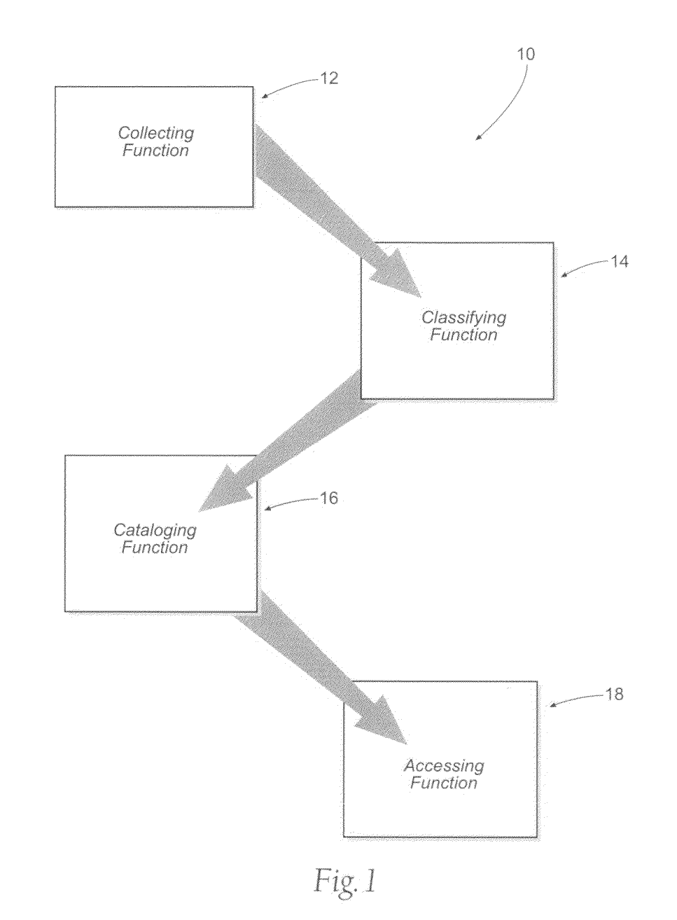

FIG. 1 shows a system 10 for collecting and classifying biological materials for storage in a repository, to make possible an informed selection by and distribution to members of the health care or clinical research communities for therapeutic and/or research purposes. As explained in greater detail below, the system 10 incorporates a series of step-wise functions to achieve its objectives.

A representative embodiment is shown in FIG. 1. In FIG. 1, four principal functions are identified. These are (i) a collecting function 12; (ii) a classifying function 14; (iii) a cataloging function 16; and (iv) an accessing function 18. The system 10 can include a lesser or greater number of principal functions, and the principal functions can be further divided into additional, ancillary or supporting sub-functions.

As described in greater detail below, the functions incorporate underlying methodologies to create an inventory of biological samples, which will also be called biosamples. The functions and their underlying methodologies link each biosample contained in the inventory to selected categories of medical, demographic, epidemiologic, genealogic, and genetic information, to make possible a robust and reliable background screening process. The functions and their underlying methodologies generate a catalog based upon the linked information and data, to aid in the identification and selection by members of the health care or clinical research communities of biosamples optimally matched with their particular research or therapeutic objectives. The functions and their underlying methodologies place the contents of the catalog in a searchable database format, augmented by an interactive interface, to streamline access to and the identification and selection of biosamples from the inventory.

A. The Collecting Function

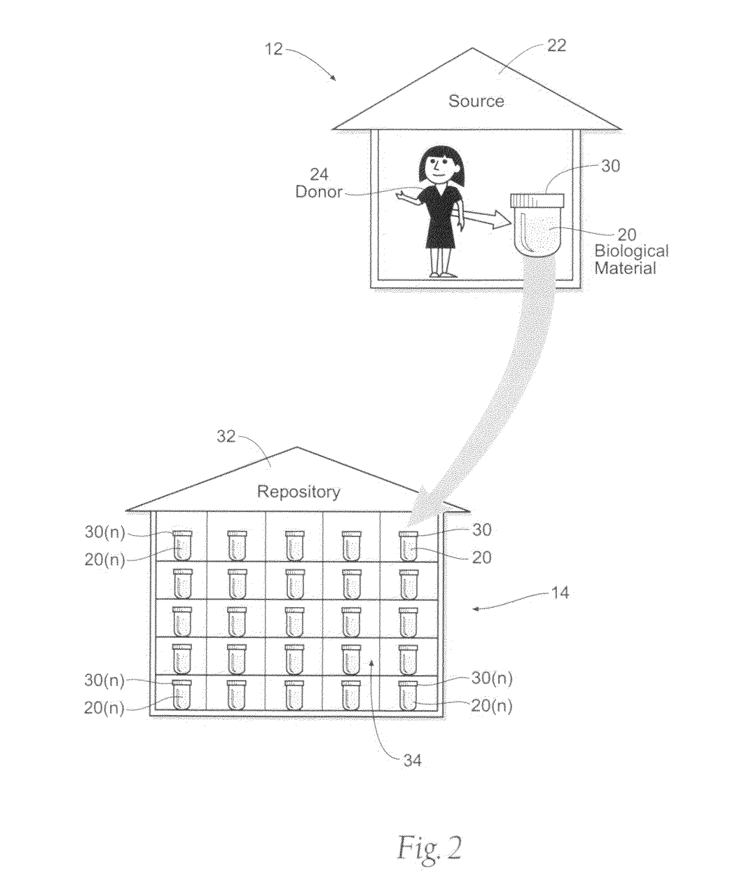

During the collecting function 12 (see FIG. 2), a given biological material 20 is collected or acquired at a source facility 22 from a donor 24 (also formally called a donor organism) for incorporation into an inventory 34 at a central repository 28. The nature of the source facility 22 can vary. For example, a given source facility can include an operating room, an outpatient clinic, a doctor's office, or specialized procurement or collection facility staffed by employees of an organization that oversees management of the system 10.

The nature of the biological material 20 can vary. Typically, the biological material 20 comprises cells, tissues, or cellular blood products from a random donor (i.e., a donor who is not the intended recipient or who is not related to the intended recipient). More specifically, the biological material 20 can comprise human stem and progenitor cells harvested from umbilical cord blood. Broadly defined, human stem and progenitor cells are unique early-stage cells which possess the capacity to transform into a multitude of cell and tissue types as they mature in a particular biologic setting or niche. For this reason, human stem and progenitor cells find use in a multitude of research and clinical applications. For example, in the field of regenerative medicine, human stem and progenitor cells can function as stand-alone therapeutic products or as components of more complex combinational tissue engineered products for tissue and organ repair, to enhance the quality and length of human life. In the field of clinical research, human stem and progenitor cells can serve to aid researchers in finding the causes of and curing disease conditions.

The biological material 20 is placed in a suitable storage container, typically in the presence of a specified preservative reagent.

The collecting function 12 desirably implements at the source facility specific protocols, forms; reagents, containers, and trained staff to meet prescribed handling and transport requirements for the biosample 20. Such requirements include, e.g., standard operating procedures (SOP's) for informed consent, labeling, and preprocessing or specific handling instructions.

The collecting function 12 may also include at the source facility a stocked inventory of reagents and containers ready to accept sterile biological materials 20 for use in both research and to be introduced back into humans, thereby requiring cGMP grade material processing.

B. The Classifying Function

During the classifying function 14, the acquired biological material 20 is received at a designated repository site 32 for storage, as FIG. 2 shows. As FIG. 2 also shows, the repository site 32 is the home of an inventory 34 of many biological materials 20(n) contained in individual storage containers 30 (n). At the repository site 32, the biological material 20 is processed by trained staff for retention or storage in the inventory 34.

1. Unique Identifier

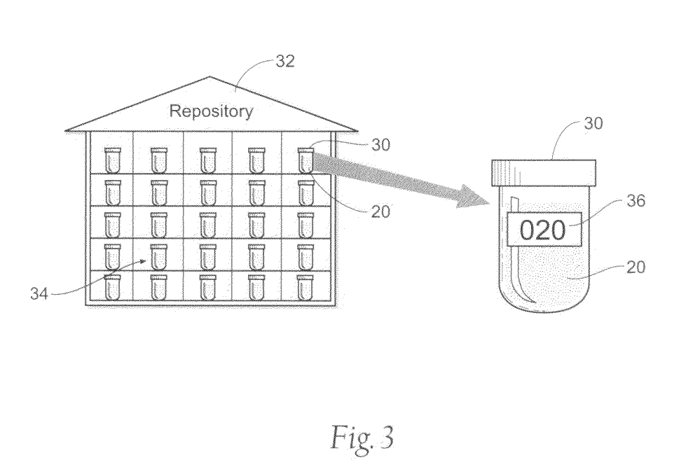

As FIG. 3 shows, the underlying methodologies of the classifying function 14, which are carried out by trained staff at the repository site 32, serve to register the type of biological material 20 contained in each container 30. The classifying function 14 assigns a unique identifier 36 to each biological material 20, so that it can be tracked within the inventory 34. The unique identifier 36 can comprise, e.g., a donor specific identification like a social security number, or a randomly assigned alpha, numeric, or an alpha, numeric, or alpha-numeric designation that is randomly assigned at the repository site and is further linked in some manner to the donor source. Each biological material 20 contained in the inventory 34 is assigned is own unique identifier 36.

The classifying function 14 carried out by trained staff at the repository site 32 can also serve to place the acquired biological material 20 into a particular condition or receptacle for storage. For example, biosamples 20 can be processed further into cell lines and/or stored in various desired ways. For example, multiwell plates containing compatible hydrogels or extracellular matrices could be seeded with cancer cells from a cancer biosample, creating a diagnostic plate for personalized testing or research purposes to evaluate certain chemotherapeutic agents or perform research assays.

2. Classification Criteria

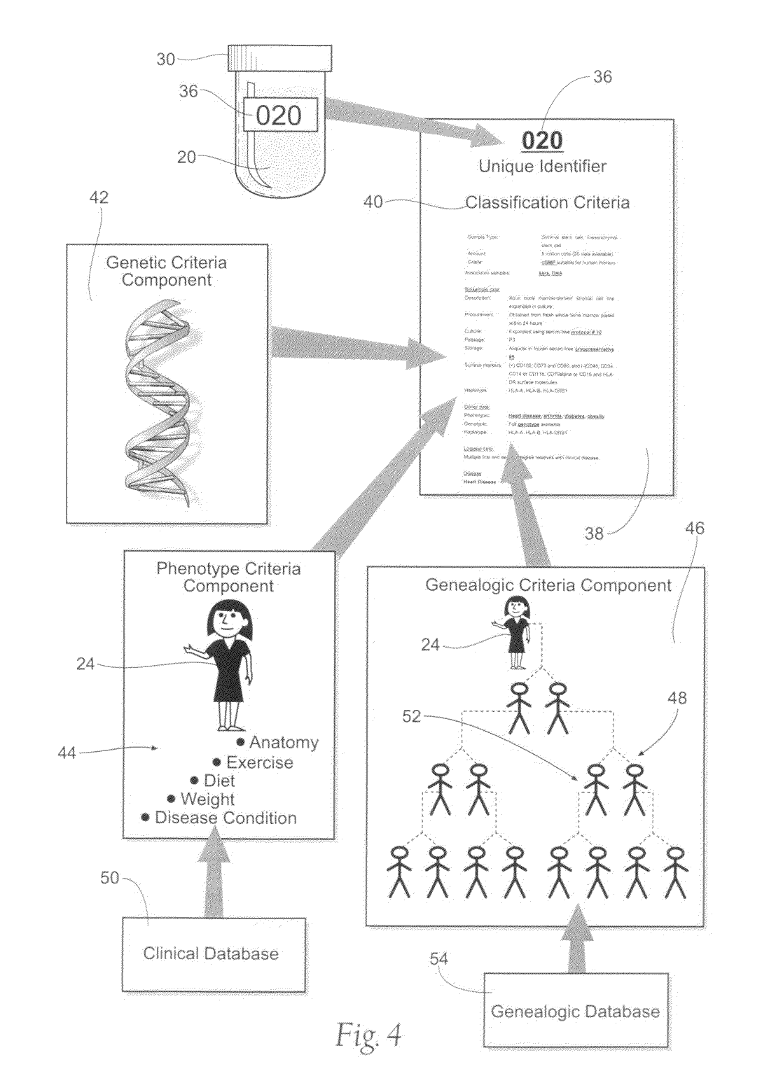

As FIG. 4 shows, during the classifying function 14, each biological material 20 entered into the inventory 34 and marked with its own unique identifier 36 is further characterized by trained staff according to pre-established classification criteria 40. The classification criteria 40 are selected to identify an array of relevant objective and semi-objective attributes of the biological material 20. The classification criteria 40 are linked to the unique identifier 36 to allow the creation of a biological material record 38 for each biosample 20. As will be described in greater detail later, the biological material record 38 with its unique identifier 36 and classification criteria 40 unique to a single biosample 20, makes it possible to generate a catalog and, desirable, an interactive, relational database, descriptive of the contents of the inventory 34.

i. Genetic Criteria Component

As FIG. 4 shows, the classification criteria 40 can include, as a relevant objective attribute, a genetic criteria component 42. The genetic criteria component 42 characterizes the genotype of the biological material 20, comprising the genetic sequence carried within the DNA of the material, biomarkers, haplotype information, etc. The genotype can be assessed by conducting appropriate tests and assays, which can be performed on a small aliquot or sample of the biological material 20.

Examples of tests and assays that can be performed include, e.g., flow cytometry analysis for surface marker phenotype, colony forming assays for potency testing, differentiation assays, and analysis of DNA using simple or complex technologies such as micro array analysis or complete genotyping and biomarker analysis. Also, certain screens for infectious disease may be performed as needed or required. The type and number of tests and assays can vary according to the type of biological material 20 and its intended field of use.

ii. Phenotype Criteria Component

The classification criteria 40 can also include, as a relevant objective attribute, a phenotype criteria component 44. The phenotype criteria component 44 includes identifying and recording observable outward traits and characteristics that are manifest in the donor organism 24, such as anatomy, diet, exercise, weight, disease conditions, and general overall health. Phenotypic information can be obtained through personal interviews, histories, and physicals. Phenotypic information can also be obtained from clinical information, such as, medical records from hospitals, other treatment centers and facilities, and their associated databases. It is believed that one important phenotype to be indentified and recorded as a phenotype criteria component 44 by the system 10 includes the donor organism's propensity to manifest a particular disease condition or conditions, e.g., heart disease, arthritis, forms of cancer, obesity, diabetes, etc.

Phenotypic information relating to a cancer disease phenotype can be obtained for certain donors from available databases 50. For example, clinical information pertaining to cancer disease patients is maintained by the Cancer Clinical Research program at the University of Utah/Huntsman Cancer Institute. This organization maintains a database (called the CCR Database) that provides a common repository for cancer specific clinical and research data to support translational research. Information in the CCR Database includes patient demographics, test results, samples, diagnosis history, staging history, and cancer progression.

Access to a database 50 such as the CCR Database during the classifying function 14 can provide important cancer phenotypic classification criteria 40 for linking to a given biosample 20. The system's inclusion of both a genetic criteria component 42 and a disease-specific phenotype criteria component 44, linked to a uniquely identified biosample 20, is significant. It is recognized that the sequencing a genome is not sufficient for prediction of phenotype. The phenotype is known to be the result of both the genotype and other “environmental factors,” which can vary greatly as an organism develops from the point of fertilization to adulthood and beyond. This distinction between genotype and phenotype allows two organisms to share identical DNA and genetic sequence, while, at the same time, their outward traits and characteristics (their phenotypes) can be quite different.

This observation in the field of genetics has lead to the widespread study of epigenetics. Epigenetics is defined as the study of heritable changes in gene function without changes in the DNA sequence. This can occur through several mechanisms such as DNA methylation, histone acetylation, and RNA interference. The resultant work of such mechanisms can be responsible for gene activation or inactivation. Why and how these mechanism are allowed to take place is still under study but forms the bases for such theories as the “nature vs. nurture” hypothesis, in which it is felt that certain genetic potential bestowed by nature can only be realized through correct environmental influence.

By identifying and registering the phenotype criteria component 44 (in particular, the propensity for the donor organism to manifest a particular phenotype) in association with a particular biosample 20 from the donor organism, the system 10 significantly increases the research and/or therapeutic value of any biosample obtained from that organism. Inherent to every cell that will be used for therapeutic purposes is a potential to introduce new genetic material to the host with the possibility of creating a new disease state, perpetuating the current disease state under treatment, or a failure of cellular therapy based on cellular ineffectiveness or lack of potency. Knowing that a donor possesses a gene for a particular trait that is readily identifiable, and obtaining a biosample from that donor and linking that biosample in a record 38 with a phenotype criteria component 44, is very useful knowledge for research and therapeutics. In research, this biosample could be used to study the genetics of this related cohort. This is especially important in a lethal or debilitating disease that should otherwise be expressed by the individual contributing the sample. In terms of therapeutics, if one is looking to produce a biologic therapy from biosamples obtained from other humans, then knowing the propensity of that biosample to manifest certain disease phenotypes is critically important to increasing the safety and effectiveness of a therapeutic product.

iii. The Genealogy Criteria Component

In the representative embodiment, as shown in FIG. 4, the classification criteria 40 are selected to also reveal additional relevant attributes of the biological material that can be considered “semi-objective” in nature. For example, in the representative embodiment, the classification criteria 40 include, in addition to the genetic criteria component 42 and a phenotype criteria component 44, a genealogy criteria component 46. The genealogy criteria component 46 identifies and records lineage information 52 pertaining to the donor organism 24 and the donor's ancestors 48. The lineage information 52 desirably includes phenotypic information pertaining to the donor's ancestors 48, and, in particular, the propensity of the donor's ancestors 48 to manifest certain disease phenotypes, or other information pertaining to patterns of genetic inheritance and/or specific genetic mutations.

Genealogic information can be obtained through personal interviews, family histories, and other sources of direct and anecdotal information pertaining to the donor and their ancestors, such as church records, newspapers, and biographies or autobiographies. Genealogic information can also be obtained from existing genealogical databases 54 that have been created by various entities.

For example, the Utah Population Database (UPDB) is a rich source of information for genetic, epidemiological, demographic, and public health studies for residents or former residents of Utah. Researchers have used this resource to identify and study families that have higher than normal incidents of cancer or other diseases, to analyze patterns of genetic inheritance and to identify specific genetic mutations. In addition, demographic studies have shown trends in the fertility transition and changes in mortality patterns for both infants and adults. The central component of the UPDB is an extensive set of Utah family histories, in which family members are linked to demographic and medical information. The UPDB also includes diagnostic records on cancer, cause of death, and medical details associated with births. The UPDB provides access to almost nine million records. These data can only be used for biomedical and health-related research, and the privacy of individuals represented in these records and confidentiality of the data is strictly protected.

The system's integration of a genealogy criteria component 46 relating to the donor and the donor's ancestors, in association with the biosample 20 itself, and further coupled to the system's identification and recording of genetic and phenotypic criteria components and 44 relating to the donor 24—create an exponential increase in the value of any biosample obtained from the donor organism. Knowing that not only a donor, but also the donor's ancestors, possess a gene for a particular trait that is readily identifiable but may be silent through several generations—obtaining a biosample from that human donor and linking that biosample with that ancestral phenotype criteria component 44, provides very useful information for research and therapeutics. In research, this biosample could be used to study the genetics of this related cohort, to determine the mechanism responsible for silencing such a gene. This is especially important in a lethal or debilitating disease that should otherwise be expressed by the individual contributing the sample. In terms of therapeutics, if one is looking to produce a biologic therapy from biosamples obtained from other humans, then knowing the propensity of that biosample to manifest certain disease phenotypes is critically important to increasing the safety and effectiveness of a therapeutic product.

With the high cost of developing regenerative therapies and the theoretic risks to the host associated with them, the system's inclusion of both objective and semi-objective classification criteria linked to the biosample provides enhanced measures to ensure both the safety and the potency of these cellular products. Incorporating background, genetic, and lineage information for these biologic products in the classifying function 14 improves both the safety profile of our future therapies and the efficiency of the research performed using these biologic products.

3. Incorporation of Classified Biosamples with Cell Therapy and/or Tissue Engineering

The linkage of a given biosample 20 to objective and semi-objective classification criteria 40, according to the classifying function 14 as just described, is well suited for incorporation with cell therapy and/or tissue engineering in the treatment of disease states that are known to have either congenital and/or acquired origins with resultant acute and/or chronic conditions.

i. Overview of Cell Therapy for Disease

Disease and clinical pathology can be categorized in many ways. A simple scheme that understandable to everyone divides disease states into “congenital” (inborn errors or defects present at birth) and acquired (those aliments which one is not born with but acquires over time).

In addition, a further useful distinction based on rapidity of onset and length of time the disease persists can be used in conjunction with congenital or acquired to describe a particular disorder. In the latter description, the term “acute” refers to diseases or disorders of rapid onset which are sometimes recoverable and other times not (this includes trauma). The term “chronic” refers to disease or injury states that persist over extended periods of time, often having a slow or insidious onset. However, as is often the case with many disease states, an acute injury, trauma, or organ failure can lead to a lifelong chronic condition.

Cell therapies will rely on different mechanisms for these different types of disease states.

(a) Acute Disease States

For example, contrary to previous hypothesis, many of the cells delivered for acute disease states do not become incorporated or take up permanent residence in the target organ undergoing treatment. It is more likely that they are playing a reparative, restorative, and/or anti-inflammatory role to help the injured cells resist apoptosis and cell death. In these types of therapies, where the cells are not expected to remain at the site of action after recovery, potency and vitality of the raw material (the starting biosample) are probably the most important characteristics. In addition, being free from transmittable disease is also critical.

However, even in these acute cases, there is often evidence, albeit at a low frequency, that some cells do incorporate into the damages target organ for the long term. Hence, it is critical that any biomaterial, be it cellular, tissue, or serum based, be appropriately screened and classified to the highest degree possible for possession or propensity of transmittable disease states and malignant transformation prior to its use as a therapeutic.

(b) Chronic Disease or Congenital Conditions

Cell therapy for the chronic disease or congenital conditions most likely represents a similar mechanism of action as that for acute therapies, but with the additional goal of actually replacing dead or dysfunctional tissue with new healthy tissue in the process. Thus, cell therapy for chronic disease therapy most likely exploits the endocrine, paracrine and cell-to-cell effects, which are critical to combating acute injury and looks to actually add new functional tissue mass that will remain vital to organ function over time. In this situation, where cells are intended to incorporate into the host and remain viable for decades or more, it is paramount that any starting biologic material be screened and classified to the fullest possible extent prior to being used for clinical therapy.

ii. Representative Disease States Possibly Treatable by Cell Therapy and/or Tissue Engineering

Representative disease states will now be identified for the purpose of illustration, which are known to have either congenital and/or acquired origins with resultant acute and/or chronic conditions. These disease states are amendable to treatment by methods that include cell therapy and/or tissue engineering that incorporates, at least in part, reliance upon biosamples classified by the classifying function 14, i.e., according to objective and semi-objective classification criteria 40, as described.

The list of disease states that are identified is by no means exhaustive. New potential therapies for these and other ailments are numerous and include both cellular and non-cellular therapeutic options.

(a) Aging

By correlating birth and death certificates, biosamples from families with propensity for early death or prolonged life can be identified and classified as such. These samples, once classified using the classifying function 14, aid in the understanding the natural aging and disease process.

### Example

Researchers have found that telomere length shortens with age, and that individuals with extraordinary longevity have longer telomeres and possible variants in their telomerase pathway and production. By obtaining and classifying biosamples using the classifying function 14 from individuals having family histories and genealogies known for longevity, one can increase the efficiency of discovery in uncovering clues to longevity and the effects of aging. Classified stem cells from such a screened population would be ideal starting raw material if one is fashioning or engineering new tissues and organs for replacement, as the stem, cells could be more resistant to the aging and disease process.

(b) Autoimmune Disease (AD)

Representative examples of systemic AD include:

(i) Rheumatoid Arthritis (RA) and Juvenile (JRA), which affects the joints; less commonly lung, skin.

(ii) Lupus—Systemic Lupus Erythematosus, which affects the skin, joints, kidneys, heart, brain, red blood cells.

(iii) Scleroderma, which affects the skin, intestine, less commonly lung.

(iv) Sjogren's Sydrome, which affects the salivary glands, tear glands, joints.

(v) Goodpasture's Syndrome, which affects the lungs and kidneys.

(vi) Polymyalgia Rheumatica, which affects the large muscle groups.

(vii) Guliiain-Barre syndrome, which affects the nervous system.

Representative examples of localized AD include:

(i) Type I Diabetes Mellitus, which affects the pancreas islets.

(ii) Hashimoto's Thyroiditis, which affects the thyroid.

(iii) Celiac Disease, Crohn's Disease and Ulcerative Colitis, which affects the gastrointestinal tract.

(iv) Multiple Sclerosis, which affects the nervous system.

(v) Addison's Disease, which affects the adrenal glands.

(vi) Primary Biliary Cirrhosis, Schlerosing Cholangitis, Autoimmune hepatic, which affects the liver and hepatobiliary system.

(vii) Temporal Arteritis and Giant Cell Arteritis, which affects arteries of the head and neck.

### Example

Most AD disorders are disabling and somewhat unpredictable in their patterns of penetration in a given population. In addition, many do not occur until later in life. Access to biosamples systematically classified according to the classifying function 14 based upon the classification criteria 40, including, e.g., a comprehensive phenotypic screen for the absence of a targeted AD disorder, e.g., for the absence of Systemic Lupus Erythematosus (SLE), supports and advances the use of biosamples in engineering new therapies for the targeted AD disorder, e.g., SLE. The cellular therapies may exploit the anti-inflammatory traits of stem and progenitor cells, as well as the incorporation of such cells permanently into the host to provide a lasting therapy.

(c) Blood and Bleeding Disorders

Representative examples of blood and bleeding disorders include:

(i) Hemophilia, which is a genetic blood disease that causes the blood to be unable to form a firm clot normally and quickly.

(ii) Hemochromatosis, which is a most often hereditary blood disorder that causes body tissue to absorb and store too much iron. The disease (which is actually many diseases) has also been known to develop as a result of dietary iron intake in sufficient quantity. Its worst effects are preventable, by early diagnosis and treatment, but, if the patient is not found in time, it is crippling and potentially fatal.

(iii) Polycythemia Vera, which occurs in one to five of every 100,000 people. With this blood disorder, patients not only have more red cells, with hematocrits that can almost double in some cases, but there is also a slow, steady buildup of white cells and platelets. Blood volume also increases, which masks the disease from the doctor while putting the patient at a higher risk of blood clots and stroke. Women under the age of 40 are at particular risk for clotting complications.

(iv) Thrombosis, which identifies when the platelets try to patch up an injury within a small blood vessel and instead completely plug it up. This plug obstructs the normal flow of blood and can result in a heart attack or stroke. Also causes deep venous thrombosis or blot clots in the large veins.

(v) Von Willebrand disease (vWD), which is the most common bleeding disorder that is found in approximately 1-2% of the U.S. population. vWD results from a deficiency or defect in the body's ability to make von Willebrand factor, a protein that helps blood clot. Although vWD occurs in men and women equally, women are more likely to notice the symptoms because of heavy or abnormal bleeding during their menstrual periods and after childbirth.

### Example

Several of the more devastating blood disorders have partial elucidation of their mechanism of action at the cellular and even genetic level. The mechanisms of action vary widely with the phenotypic results manifest from a slight propensity to bleed more then other individuals to being extremely high risk for a fatal pulmonary embolus (PE). When a clinician screens biosamples for transplantation that have the ability to reconstitute either a part or all of the recipients blood (i.e. bone marrow transplant for leukemia), it is desired that the biosample not transfer a bleeding or clotting disorder in the process. The classifying function 14 makes possible the screening for known bleeding and clotting defects at the genetic or biomarker level, and thereby provides a higher level of security for the biosample recipient. For example, absent access to a biosample systematically classified according to the classifying function 14 in the treatment of a thromboembolic disease, an individual could be cured from a leukemia, but end up with a lethal clotting disorder that may otherwise go undetected until the fatal event, such as stroke or PE. Appropriate biosample background genetic, genealogy and phenotypic screening according to the classifying function 14, can avoid this undesirable scenario.

(d) Cardiovascular Disease

Representative examples of cardiovascular diseases include:

(i) Hypertension, which is the most common risk factor for heart and kidney diseases and stroke.

(ii) High Blood pressure, or hypertension, which is defined in an adult as a systolic pressure (top number) of 140 mm Hg or higher and/or a diastolic pressure (bottom number) of 90 mm Hg or higher. Blood pressure is measured and noted in millimeters of mercury (mm Hg).

(iii) Coronary Artery Disease (CAD), which is a chronic illness in which the coronary arteries, the vessels that supply oxygen-carrying blood to the heart, become narrowed and unable to carry a normal amount of blood. Most often, the coronary arteries become narrowed because of atherosclerosis, a process in which fatty deposits called plaque build up on the inside wall of an artery. Hereditary factors may also increase the risk for the disease.

(iv) Stroke: The risk of stroke is greater if a parent, grandparent, sister or brother has had a stroke. Also, African Americans have a much higher risk of death from a stroke than Caucasians do. This is partly because African Americans have higher risks of high blood pressure, diabetes and obesity.

(v) Peripheral vascular Disease: Those with strong family history of heart disease and stroke are greater risk for peripheral vascular disease.

### Example

Cardiovascular disease is the leading cause of death worldwide. There are many examples where both clinicians and researchers could help benefit patients with biosmaples extensively screened and selected according to the classifying function 14. For a researcher studying CAD, having access to biosamples from families with severe CAD increases the chance of meaningful biomarker discovery through array analysis. In the therapeutic case, the classifying function 14 offers to researchers and physicians engaged in tissue engineering the ability to grow new cardiac tissues for integration into a failing heart riddled with dysfunctional tissue. The starting raw material, whether it is cells or whole tissue, can be screened and classified according to the classifying function so that the biosample has the least propensity to fail in a particular organ, this case, the heart. Conversely, the classifying function 14 also makes possible the selection of biosamples with positive phenotypes and attributes, such as enhanced cardiopulmonary reserve correlating with the ability to perform significant exercise into the later decades of life.

(e) Cancer

Representative examples of cancer include:

(i) Breast: It has been estimated that about 5 to 10 percent of all female breast cancer cases are hereditary. BRCA1 and BRCA2 are genes involved in cell growth, cell division, and repair of damage to DNA. DNA damage occurs when a spelling error is made in the gene sequence. A changed BRCA gene can cause DNA damage in cells to go unrepaired, which increases the chance that cancer will occur. People with BRCA mutations thus may get cancer at an early age, they may develop breast cancer in both breasts, or they may develop more than one type of cancer, e.g., cancers of both the breast and the ovary. The most common type of cancer linked to BRCA1 and BRCA2 changes is breast cancer, but mutated forms of BRCA genes are linked to other cancers as well. For example, men with BRCA2 mutations are at increased risk of getting prostate cancer.

(ii) Ovarian: It has been estimated that about 5 to 10 percent of all ovarian cancer cases are hereditary.

(iii) Colon: Though most cases occur sporadically, it is estimated that 5 to 10 percent of all colorectal cancers are explained by a specific genetic susceptibility. A person who is diagnosed with colorectal cancer and who has a family history of the disease is more likely to have inherited a cancer gene than a person with no family history of colorectal cancer.

(iv) Prostate. Recent studies have concluded that a susceptibility to prostate cancer can be inherited. It estimated that 5 to 10 percent of all prostate cancer cases are considered hereditary. This means that in some families, a genetic predisposition to develop prostate cancer can be passed down from parent to child.

(v) Leukemia: Often referred to as cancer of the Blood, a malignant condition affecting the immature blood-forming cells in the bone marrow.

(vi) Thalassemia, which is a group of fatal genetic blood disorders. The world health organization (WHO) recognizes Thalassemia as the most prevalent inherited genetic Blood disorder in the world. An estimated 2 million Americans are carriers of the genetic trait for thalassemia, predominately those of Mediterranean and Asian Indian, South Asian and Chinese ancestry.

### Example

In support of the use of any biosample for therapeutics, the classifying function 14 provides the ability to perform an extensive background screen for cancer. The classification function makes possibility the creation of a large repository of biosamples known to carry cancer, which is extremely valuable for biomarker discover, therapeutic testing, and creation of new disease models of cancer. It provides a very important component of recovery and handling of biosamples from cancer patients or families with cancer phenotypes, including the ability to culture primary cell lines from individual tumors and test them for susceptibility to current and new therapeutics. Researchers will find the level of information that can be made available and linked to cancer biosamples according to the classifying function of great value in studying the behavior of certain cancers, both as a broad category and at the personalized level where individual tumors and cancers will respond differently to manipulation and therapy.

(f) Diabetes

Both Types I and II diabetes can run in families, and there is a genetic component to the diseases which may, or may not, be inherited. The majority of diabetics of either type do not have a first degree relative with the disease. Still, the genetic predisposition to diabetes is fairly easy to come by. In both cases, there are probably multiple contributing genetic locations and the probability that one will get the disease depends on which subset of these affected locations they posses. There also appear to be some genes that protect individuals from the disease. Especially with Type II diabetes, phenotype along with multiple epigenetic, demographic and psychosocial factors plays a significant role in whether an individual acquires and manifests the disease.

### Example

One example of cell therapy for Diabetes is implantation of tissue engineered cultured pancreatic-like cells (i.e. pseudo beta islet cells engineered to release insulin in response to the body's needs). There are multiple candidate cells under study to suit this type of manipulation. The classification of raw biosamples (cells and pancreatic tissue) from non-diabetic donors or from those without a history of a pancreatic cancer or disorder according to the classifying function will be critical in creating a master cell bank of insulin producing cells that will need to function for decades inside the recipient.

(g) Neurology/Neurodegenerative Disorders

Cerebral palsy (CP) is a broad term that describes a group of neurological (brain) disorders. It is a life-long condition that affects the communication between the brain and the muscles, causing a permanent state of uncoordinated movement and posturing. CP may be the result of an episode that causes a lack of oxygen to the brain.

Parkinson's disease, Huntington's disease, Alzheimer's disease, and Amylopic Lateral Sclerosis (ALS) represent other neurological disorders for which effective treatment is yet to be developed, but where cell therapy and regenerative medicine hold great promise.

### Example

Many of the neurological disorders amenable to cell therapy in the future will require lasting residence of the implanted cells. These cells may be minimally manipulated or maximally genetic engineered to produce a particular enzyme, cytokine or bioactive substance to treat the manifestation of the particular disease (i.e. tissue engineered dopamine producing neural progenitor cells for Parkinson's Disease). The quality of the raw material will be paramount, with a clean phenotypic history of degenerative brain disorders being required. Since many of these disorders manifest later in life, the classifying function 14, including an extensive review of genealogy records linked to medical data, will yield the best screen for this type of raw material supply.

(h) Ophthalmology

Hereditary ophthalmologic disorders may be isolated (only affecting the eye) or part of a syndrome when associated with other physical findings. Isolated or non-syndromic eye disorders can be inherited in a family in a number of different ways, with the risk for unaffected family members to have a child with an eye disorder being dependent upon the pattern of affected individuals within the family. Examples of non-syndromic hereditary eye disorders include: microphthalmia, anophthalmia, strabismus, and congenital glaucoma. Syndromes that involve eye disease as a component of the condition include: blepharophimosis-ptosis-epicanthus-inversus syndrome (BPES), oculocutaneous albinism, Marfan syndrome, Stickler syndrome, and CHARGE (coloboma, heart anomalies, atresia of the choanae, retardation of growth and development, genital/urinary anomalies, ear abnormalities or deafness) syndrome. Several isolated eye disorders and genetic syndromes with an ophthalmologic component now have identifiable gene mutations known to cause disease. Ophthalmologists are often on the “front line” in evaluating individuals and families with such conditions. A greater knowledge of the clinical and molecular features of these disorders is important for accurate diagnosis, appropriate genetic counseling, and application of treatment strategies targeted at the individual's diagnosis and genetic status.

### Example

Cell therapy for ophthalmology is already under extensive study both at the pre-clinical and clinical stage. Disorders such as macular degeneration (MD) are being experimentally treated with stem cells by exploiting their reparative and anti-inflammatory effects. Whether these implanted cells need to become permanent residents of the retina is still unclear. However, use of cells that have a clean phenotype for MD and other ophthalmologic disorders is ideal for these types of therapies where some cells may incorporate and others may be transient soldiers. Also, in the case of corneal disease and injury, cell therapy will play a great role in the future. The classifying function 14 makes possible the collection of and systematic access to tissue engineered corneal cells and whole tissues provided in an off-the-shelf allogeneic form. In this respect, the classifying function 14 can serve to redefine the treatment of several of the most common blinding disorders and injuries to the cornea. The classifying function 14, including tertiary phenotypic, genotypic screening will be a critical component for the safety and reliability of these therapies and products.

(i) Orthopedics

In addition to Juvenile Rheumatoid Arthritis (JRA) and cancers of the bone such as Ewings Sarcoma and Osteogenic Sarcoma there are many congenital and hereditary orthopedic conditions, which include Clubfoot, Congential Limb Defects, Developmental Dysplasia of the Hip, Metatarsus Addusctus, Muscular Dystrophy, and Osteogenesis Imperfecta. Also rheumatoid arthritis (RA) and degenerative joint disease (DJD) are very common orthopedic disorders resulting in the destruction of bone and joints. Together these latter two disorders are responsible for huge health care expenditures each year. Their mechanisms of action and origin as congenital, acquired and/or epigenetic still remains unclear.

### Example

Orthopedics along with cardiovascular disease is one of the next two likely fields to see cell therapy become standard of care in the clinics. In the case of bone and joint repair, clinicians are already using bone marrow derived stem cells to speed healing of bone defects and cultured knee meniscus biopsies for repair of worn out joints, respectively. Although these therapies today are often autologous, clinicians will likely be delivering an off-the-shelf allogeneic product in the near future. The classifying function 14 including the screening of donors of the raw tissue and cellular material for disorders such as RA and DJD, provides another level of confidence to the durability of cells implanted intended to last decades or even a lifetime.

(j) Renal Disease

Examples of renal disease include

(i) Polycystic kidney disease (PKD), which is a genetic disorder in which many cysts grow in the kidneys. PKD cysts can slowly replace much of the mass of the kidneys, reducing kidney function and leading to kidney failure.

(ii) Hereditary Nephritis (Alport's syndrome), which is a genetic disease characterized by the onset of hematuria in early childhood and later progression to renal failure, predominantly in males, accompanied by the development of sensorineural (high frequency) hearing loss. A typical male patient presents with the onset of persistent gross or microscopic hematuria, sometimes exacerbated by upper respiratory illness, before the age of six.

(iii) Fanconi Syndrome, which is a dysfunction of the proximal tubule which gives rise to excessive urinary losses of amino acids, glucose, phosphate, bicarbonate, electrolytes, and other solutes. These losses lead to the clinical problems observed in this syndrome, e.g. acidosis, dehydration, rickets, and growth failure.

(iv) Cystinosis, which is the most frequent cause of renal Fanconi syndrome in children. First describe in 1903, it was then readily confused with cystinuria, a disorder with defective tubular reabsorption of cystine, arginine, lysine, and ornithine.

(v) Nephrogenic Diabetes Insipidus, do to which the kidneys fail to respond to the ADH and do not reabsorb more water. The hereditary form of NDI occurs largely in males, and is thought to be linked with variable penetrance. Manifestations occur shortly after birth, typically with polyuria and polydipsia. Because of the young age at presentation, the polyuria may not be recognized, and dehydration may ensue. Symptoms such as irritability, poor feeding, and poor weight gain may develop. Fever develops in some infants, secondary to dehydration. Encephalopthy may develop, secondary to repeated damage from dehydration and hypernatremia. Often, there is psychomotor retardation, and learning deficits.

### Example

Renal failure and complications of other disorders resulting in acute renal failure (ARF) represent one of the largest health care expenditures in the world today. Much of this renal compromise is due to ischemic injury secondary to low flow states related to sepsis and also due to circulating toxic cytokines that are injurious to the kidney during states of multi-organ stress or failure. Ongoing cell therapy trials are underway with off-the-shelf allogeneic bone marrow derived mesenchymal stem cells (MSC) and are showing great promise in the treatment of the acute kidney injury. It is felt that these MSCs are transient and short-lived when used for the treatment of ARF but may rely on the vitality of the donor for maximal benefit. The classifying function 14, including extensive background screen for the healthiest donors with the most resilient phenotype, provides an enhancement to this therapy. In the situation of chronic renal failure (CRF), however, delivered cells may actually need to incorporate and take up permanent residence in the injured kidney parachyma in order to restore function. The classifying function 14 makes possible maximal background screen for the lack of propensity to both ARF and CRF, and will provide the safest master cell bank used for therapy.

C. The Cataloging Function

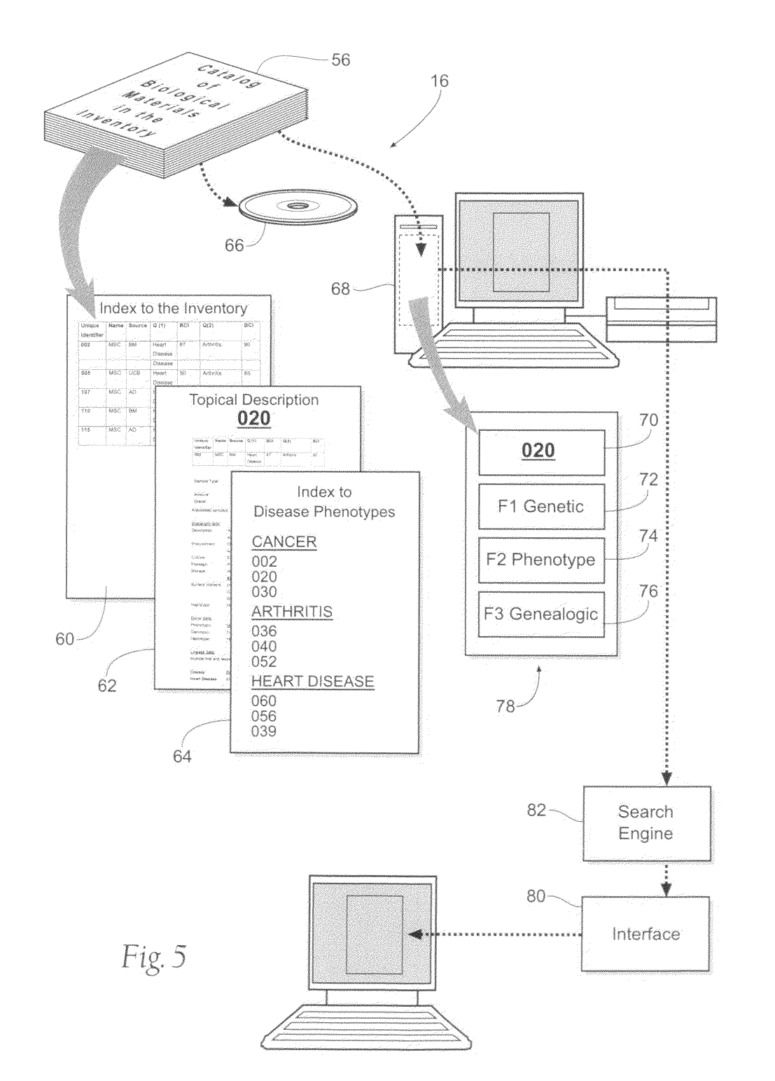

In the illustrated embodiment (see FIG. 5), the system 10 further includes a cataloging function 16. The cataloging function 16 intuitively links the unique identifier 36 of each biosample 20(n) in the inventory 34 with the array of diverse information assembled on the biological material record 38, including the classification criteria 40 identified and recorded for the biosample 20 (n), to create a catalog 56. The catalog 56 organizes the records 38 of the biosamples 20(n) contained the inventory 34 to provide one or more helpful “helicopter views” of the inventory 34, identifying to prospective users or customers the biological materials 20(n) present in the inventory 34 according to their genetic, phenotypic, clinical, and genealogic attributes.

1. The Catalog

As shown in FIG. 5, the catalog 56, e.g., can be embodied in the form of a printed medium or booklet 58. In its most simple format, the printed catalog booklet 58 can include an index page 60 to the inventory 34 arranged by unique identifier number, providing in effect a table of contents to the inventory 34. The printed catalog booklet 58 can also include one or more topical pages 62, individually dedicated to each biosample 20(n). Each topical page 62 lists for the respective biosample 20(n), the classification criteria 40 identified and recorded for the biosample 20(n).

In more expansive formats, the printed catalog booklet 58 can also include one or more classification index pages 64. These pages 64 are arranged according to one or more specific classification criteria 40, listing for the specific classification criteria 40 the biosamples 20(n), arranged by unique identifier, manifesting the specific classification criteria 40. For example, in FIG. 5, the classification index page 64 is dedicated to listing by disease phenotype (e.g., cancer, arthritis, etc.) those biosamples 20 (n), arranged by unique identifier, manifesting the disease phenotypes, thereby providing an index to the biosamples 20(n) having the propensity for the donor organism to manifest particular disease phenotypes or, alternatively, the absence thereof.

Using a printed catalog booklet 58, indexed according to classification criteria, a person can select biologic material for use in studying a particular disease state (research application), or select biologic material to treat a human in a therapeutic manner for a particular disease state (clinical application).

As FIG. 5 also shows, the catalog 56 can also be recorded and distributed in a digital medium 66, such as a CD or DVD. The contents of the catalog 56 can also be made available through an internet web page.

Alternatively, or in combination with a printed indexed catalog, the catalog 56 can be incorporated into a relational database program 68 that dynamically links each biosample 29(n) to its classification criteria 40. In the relational database 68, each biosample (identified by its unique identifier) represents an entry 70, and each classification criteria 40 associated with the biosample 20(n) represents a field—Field 1, Field 2, and Field 3, respectively for Genetic Criteria Field 72; Phenotype Criteria Field 74; and Genealogy Field 76, The multiple fields and the classifying criteria are electronically grouped together by the database 68 as an electronic counterpart to the biological material record 38, discussed above. Thus, within the catalog database 68, each entry 70 (comprising a biosample identified by its unique identifier) contains multiple fields 72, 74, and 76 (comprising the classification criteria 40), which are grouped together as a record 78. From the database 68, printed catalogs 58 can be generated tailored to specific requests. Individual records within the database can be printed, or indexes can be created based upon fields common to different records.

The cataloging function 16 serves to warehouse and maintain the diverse data associated with the inventory 34 of biosamples 20(n). The catalog 56 therefore serves a valuable screening function consistent with the demand for new and innovative cellular therapies. Many of these therapies address specific disease states, which are known to have a hereditary disposition or an identified genetic factor. The source of therapeutic “product” being delivered to the patient will often be harvested from an unrelated human donor. Thus, much of the success of these cellular interventions will rest on the integrity of the donor material or cellular composition, which includes overall genetic makeup, genotype, phenotype and lineage characteristics. The cataloging function 16 makes it possible to screen cellular and tissue biosamples for the presence or absence of a disease state. Thus, one can make an educated decision made as to the probability that a particular biosample will be useful for the study or treatment of a particular disease state. Expanding the screening process of biosamples by integrating extensive lineage information found in databases like the Utah Population Database enhance the power and effectiveness of the screening process the system 10 provides. The cataloging function 16 provides a resource from which parties who are, e.g., looking for a biological sample to study a particular disease condition, or looking for a biological sample for use as a raw material to develop a therapeutic biologic product, can systematically locate and select from an inventory index a particular biological sample that will best meet their particular research or therapeutic objectives.

As will be discussed in greater detail later (and as FIG. 5 shows), the accessing function 18 takes the screening provided by the cataloging function 16 a step further, by the incorporation of an interactive interface 80 with a search engine 82 to the catalog database program 68. In this way, the accessing function 18 makes it possible for a user or customer, by classifying of focused advanced, multiple-part inquiries or queries, to better identify and select a biological sample in the inventory that is best matched with their particular research or therapeutic objectives.

2. The Biosample Criteria Index

The catalog functioning 16 desirably includes a correlating function 84 that correlates and statistically weighs the classification criteria 40 for a given biosample 20(n) to derive a Biosample Criteria Index (BCI) 86. The BCI 86 can comprise, e.g., a dimensionless, numeric quantity. The magnitude and/or sign (positive or negative) of the BCI 86 reflect the propensity of the donor organism, from which the biological material is collected, to manifest a particular selected phenotype and/or genotype.

The correlating function 84 purposefully selects and weighs the appropriate classification criteria 40 according to statistical, clinical, and/or anecdotal considerations according to preprogrammed rules 88 to derive the BCI. The preprogrammed rules 88 for deriving the BCI 86 can, e.g., utilize biostatistical analysis to leverage the combined information in the classification criteria 40 to arrive at an index expressing a confidence interval possessed in the background of each biosample 20(n). The BCI can be calculated in real time or as needed for the most common disease processes, or for less common processes, upon request or upon availability of the data.

The preprogrammed rules 88 are developed to generate a BCI 86 that will, within a confidence interval, reliably inform a prospective user or customer the likelihood that a particular phenotype and/or genotype is present (or absent) in a given biosample 20(n). The nature and relevance of the classification criteria 40 selected, and the manner in which the preprogrammed rules 88 operate to correlate and weigh that data, determine the statistical confidence interval of the BCI 86 to predictive of the presence (or absence) of the selected genotypic and/or phenotypic information in the donor organism.

Desirably, a selected phenotype can comprise the presence or absence in the donor organism of a given disease condition or combination of disease conditions, e.g., heart disease and/or arthritis and/or cancer and/or obesity. For example, in the representative example, the classification criteria 40 are selected to reveal functional manifestations inherent in the particular biological material as they relate to a desired phenotype; namely, in the representative embodiment, the disease condition or conditions of interest. A numerically high (or positively signed BCI 86) indicates the higher likelihood that the given disease condition or combination of disease conditions is manifest in the biosample 20(n). Conversely, a numerically low (or negatively signed BCI 86) indicates the lower likelihood that the given disease condition or combination of disease conditions is manifest in the biosample 20(n).

Like the classification criteria 40, the BCI 86 for each biological material in the inventory is desirably included in the biological material record 38, and the electronic counterpart record 78 in the catalog database program 68. As such, the BCI 86 can be linked to the unique identifier assigned to the biological material 20(n) in the catalog 56, whether in printer or electronic form. In this way, the cataloging function 16 develops a universe of interlinked information and data that can be manipulated to indicate relationships between a uniquely identified sample, the classification criteria 40, and the BCI 86. In the relational catalog database program 68, the BCI 86 can comprise an additional field 90 in each electronic biosample record 78. The BCI 86 is determined through a systematic information collection process, drawing upon different types of classification criteria 40, which are collected based upon different analytical methods and using different resources and databases. It is the specific combination of these methods and resources that make the BCI 86 unique from other processes and methods aiding the selection of biosamples for research and therapy purposes. Databases included in the determination of a biosample's BCI 86 are clinical databases maintained by hospitals and other treatment centers and facilities, and genealogy databases such as the Utah Population Database (UPDB). Also, extensive genetic information either already exists or can be obtained from the biosample itself and/or from a variety of procedures, many of which are very minor. Phenotypic information can be obtained through personal interviews, histories and physicals and clinical information such as medical records.

The derivation of a BCI 86 based on diverse genealogic, genetic, phenotypic and clinical data allows a discrete dimensionless number to be assigned to a biosample sample 20(n), which is representative of the presence or absence of genotypic and phenotypic information. Biosamples 20(n) linked to such diverse, extensive classification criteria 40 and further characterized by a BCI 86, can be specially identified to prospective users or customers by a unique designation, e.g., as LineaCells. The designation LineaCells identifies a biosample 20(n) that has been specially screened and characterized for use both as a research reagent to determine answers to scientific questions, as well as a raw material for therapeutic biologic products.

D. The Accessing Function

The accessing function 18 includes the interactive selection by a prospective user pr customer of a particular biosample 20(n) from the inventory 34 best matched to their research or therapeutic needs. The preceding collection, classifying, and cataloging functions, respectively 12, 14, and 16 provide an information-rich infrastructure for the biosample inventory 34 that makes highly informed selection possible.

1. The Interactive Interface to the Catalog

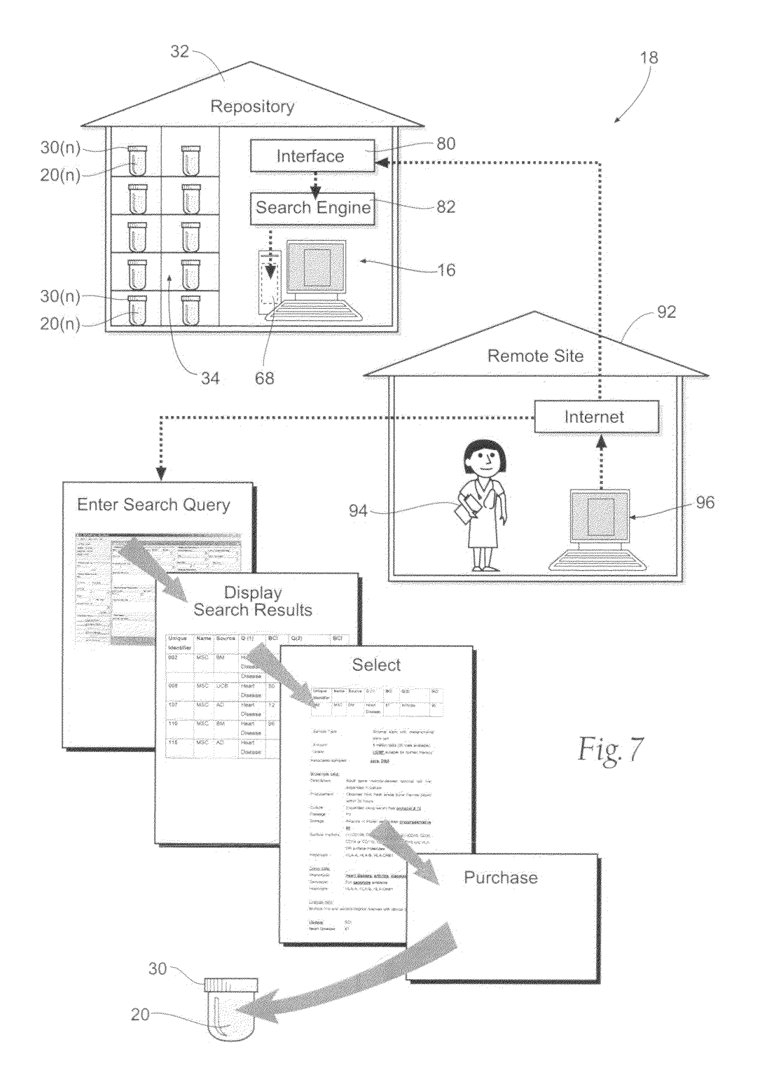

The system 10 creates hard linkage between a given biosample 20(n) and extensive amounts of data, both objective in terms of definable phenotype and genetic information, and semi-objective in terms of case clinical descriptions and clinical data derived from medical records, as well as genealogic data from various sources. By design, the system 10 links to a given biosample 29(n) much more data then a typical single user or customer could ever use. It is for this reason that, in addition to the actual data being warehoused and maintained by the classifying and cataloging functions 14 and 16, the accessing function 18 includes the interactive interface 80 with the catalog database program 68, including a robust search engine 82 (see FIGS. 5 and 7) to make it very easy for a prospective user or customer to filter out which biosample or biosamples are most appropriate for a particular indication.

Because the information available to the user or customer will be expansive, the accessing function 18 desirably keeps the catalog database program 68 online in secure servers at the repository site 28, with coded access by a prospective user or customer 94 at a remote site 92. The prospective user or customer 94 at the remote site 92 will only see and experience via a remote workstation 96 a user friendly interface or web portal, providing an overview view of the contents of the inventory 34, indicating what biosamples 20(n) are held, and what data are available online or by request through staff assistance. The prospective user or customer 94 will have access only to de-identified data (i.e., data from which the particular personal identity of the donor or other individuals have been de-linked).

The technical feature of an online searchable interface 80 that the accessing function 18 provides, linked by the cataloging function 16 to genetic, phenotypic, clinical, and genealogic data sourced from multiple sources, including different types of databases, is unique and justifies use of the special designation “LineaCells” to the associated biosamples, as previously discussed. This technical feature of the system 10 adds great value to the “LineaCells” biosample, by eliminating time consuming individual manual searches and rudimentary testing of biosamples after collecting by the user. The presence of the BCI 86 in the catalog 56, or its electronic counterpart 68, derived from multiple arms of information and also accessible by use of the interface 80, is also another unique technical feature of the system 10.

The accessing function 18 provides the ability to identify, select, retrieve, package and deliver a biosample 20(n) to a user or customer in a useful way.

Initial level of data will include the objective classification criteria 40, comprising, e.g., biosample-specific data, or those data specific to the physical sample or donor organism itself. Such objective classification criteria 40 data includes anatomical, phenotypic, genetic, and functional data, as already discussed. Other levels of data linked to the sample can come from the other semi-objective classification criteria 40 linked to the biosample, which are derived from other sources, including multiple databases, such as but not limited to clinical records, population and lineage databases such as the Utah Population Database.

In addition to the online search, once users find the biosamples of interest, the accessing function 18 provides access to further information that may need actual human assistance to drill down to the additional information requested. Some of this information may be behind a HIPPA or proprietary “firewall” requiring LineaCell staff access only. This information can be “scrubbed” by LineaCell staff and delivered to the user in a compliant fashion or alternatively the LineaCells staff can assist the user in obtaining the appropriate IRB approvals to access the data as part of the design of a specific research project. This type of custom advanced data search and collecting assistance also adds great value, offering the user a boutique search service not available elsewhere.

### Example of Use of the Interface

For illustrative purposes (see also FIG. 7), a sample query and results are presented below. Words highlighted in BOLD below signify “clickability” leading to next level of information associated with item via hyperlink. The next level may also include a definition of protocol or process.

A prospective customer logs into the website where the catalog of biosamples is found. The prospective customer can search the entire catalog on line based on a query that specifies a desired biosample type. Using the interface, the potential customer enters any of the typical description qualifiers that would be associated with any given sample (e.g., tissue type; cell type; cancer specimen, associated disease process, etc. . . . ), as follows:

### Example Search Query

### Adult Mesenchymal Stem Cells and Heart Disease and Arthritis

The search engine 82 searches the relational links established in the catalog database program 68. The search engine 82 yields a list of “hits” correlating to the query, which the interface displays. Both biosamples linked to a BCI (i.e., LineaCells) and other biosamples not linked to a BCI can be listed, depending upon preference of the user. Returned with each hit will also be the BCI, the magnitude of which indicates the propensity of disease associated with the biosample. By clicking on the hits of interest, the prospective customer will see a more detailed description of the biosample.

### Example Query Return

### LineaCells (with BCI)

| Unique | | | | | | |
|---|---|---|---|---|---|---|
| Identifier | Name | Source | Q (1) | BCI | Q (2) | BCI |
| 002 | MSC | BM | Heart Disease | 87 | Arthritis | 90 |
| 026 | MSC | BM | Heart Disease | 91 | Arthritis | 86 |
| 005 | MSC | UCB | Heart Disease | 50 | Arthritis | 65 |
| 107 | MSC | AD | Heart Disease | 12 | Arthritis | 50 |
| 110 | MSC | BM | Heart Disease | 96 | Arthritis | |
| 115 | MSC | AD | Heart Disease | | Arthritis | 97 |

### Example Query Return

### Other Biosamples

| Unique Identifier | Name | Source | Q (1) | BCI | Q (2) | BCI |
|---|---|---|---|---|---|---|
| 020 | MSC | BM | | NA | | NA |
| 032 | MSC | AD | | NA | | NA |
| 033 | MSC | UCB | | NA | | NA |

Abbreviation Codes: BM—Bone Marrow
AD—Adipose
UCB—Umbilical Cord Blood

The prospective customer may then click on one or more biosamples to compare prior to purchase, as follows:

| Unique | | | | | | |
|---|---|---|---|---|---|---|
| Identifier | Name | Source | Q (1) | BCI | Q (2) | BCI |
| 002 | MSC | BM | Heart Disease | 87 | Arthritis | 90 |
| 026 | MSC | BM | Heart Disease | 91 | Arthritis | 86 |

The prospective customer may Double Click on a Biosample to see expanded description and List of Data available, as follows:

### Sample Selection #1

| Unique | | | | | | |
|---|---|---|---|---|---|---|
| Identifier | Name | Source | Q (1) | BCI | Q (2) | BCI |
| 002 | MSC | BM | Heart Disease | 87 | Arthritis | 90 |

Sample Type: Stromal stem cell, mesenchymal stem cell
Amount: 5 million cells (20 vials available)
Grade: cGMP suitable for human therapy
Associated samples: sera, DNA

### Biosample Data:

Description: Adult bone marrow-derived stromal cell line expanded in culture.
Procurement: Obtained from fresh whole bone marrow plated within 24 hours
Culture: Expanded using serum-free protocol #10
Passage: P3
Storage: Aliquots in frozen serum-free cryopreservative #5
Surface markers: (+) CD105, CD73 and CD90, and (−) CD45, CD34, CD14 or CD11b, CD79alpha or CD19 and HLA-DR surface molecules
Haplotype: HLA-A, HLA-B, HLA-DRB1

### Donor Data:

Phenotypic: Heart disease, arthritis, diabetes, obesity
Genotypic: Full genotype available
Haplotype: HLA-A, HLA-B, HLA-DRE1

### Lineage Data:

Multiple first and second-degree relatives with clinical disease:

| Disease | BCI |
|---|---|
| Heart Disease | 87 |
| Arthritis | 90 |
| Diabetes | 80 |
| Obesity | 50 |
| Cancer | 95 |

### BS #002 Heart Disease BCI 87

5 first-degree relatives with documented clinical disease
12 second-degree relatives with documented clinical disease

### BS#002 Arthritis BCI 90

2 first-degree relatives with documented clinical disease
5 second-degree relatives with documented clinical disease

### BS #002 Diabetes BCI 80

3 first-degree relatives with documented clinical disease
5 second-degree relatives with documented clinical disease

### BS#002 Obesity BCI 50

5 first-degree relatives with documented clinical disease
15 second-degree relatives with documented clinical disease

### BS #002 Cancer BCI 95

5 first-degree relatives with documented clinical disease
20 second-degree relatives with documented clinical disease

### Sample Selection #2

| Unique | | | | | | |
|---|---|---|---|---|---|---|
| Identifier | Name | Source | Q (1) | BCI | Q (2) | BCI |
| 026 | MSC | BM | Heart Disease | 91 | Arthritis | 86 |

Sample Type: Stromal stem cell, mesenchymal stem cell
Amount: 5 million cells (100 vials available)
Grade: research use only
Associated samples: NA

### Biosample Data:

Description: Adult bone marrow-derived stromal cell line expanded in culture.
Procurement: Obtained from fresh whole bone marrow plated within 24 hours
Culture: Expanded using standard laboratory protocol #8
Passage: P5
Storage: Aliquots frozen in standard cryopersative #3
Surface markers: (+) CD105, CD73 and CD90, and (−) CD45, CD34, CD14 or CD11b, CD79alpha or CD19 and HLA-DR surface molecules
Haplotype: NA

### Donor Data:

Phenotypic: Heart disease, arthritis,
Genotypic: Partial genotype available
Haplotype: NA

### Lineage Data:

Multiple first and second-degree relatives with clinical disease:

| Disease | BCI |
|---|---|
| Heart Disease | 87 |
| Arthritis | 90 |

### BS #026 Heart Disease BCI 87

4 first-degree relatives with documented clinical disease
6 second-degree relatives with documented clinical disease

### BS#026 Arthritis BCI 90

4 first-degree relatives with documented clinical disease
5 second-degree relatives with documented clinical disease

The query return expressing the BCI can include a computer generated diagram of a lineage tree with colored dots for (−) (+) or unknown disease correlating to what records and data are available.

Upon clicking a documented hyperlink, the user can either see a list of de-identified people with a description of their particular disease manifestation or they could be prompted to contact LineaCell staff for further release of data that may require more sensitivity or higher security.

Upon determining the appropriate sample for a specific need, the prospective customer can add the selected biosample or samples to a web cart and electronically check out. Checkout will work similar to most online merchandise purchases where a variety of payment methods are accepted. A material transfer agreement may apply to certain biosamples in addition to other proprietary forms and agreements that may need to be filled out.

Once purchase transaction is completed, the customer will be given the appropriate biosample access code to continue to work with LineaCell staff to obtain data linked to the sample.

The LineaCell staff will ship the biosample with minimal paper information attached; essentially just the required labeling, handling and shipping information. This will increase the security of the information transferred to the user.

E. Integration of Genealogy Criteria with Routine Patient Monitoring/Diagnosis

The classification criteria 40 described in connection in the revelation of relevant attributes of a biological material can also be integrated by healthcare providers into their routine care and treatment of an individual patient. In addition to the routine compilation of objective health-related phenotypic information for a given patient (such as, e.g., anatomy, diet, exercise, weight, disease conditions, and general overall health), obtained e.g., through personal interviews, histories, and physicals, genealogy attributes drawn from lineage information pertaining to the patient's ancestors can also be compiled. The lineage information desirably includes phenotypic information pertaining to the patient's ancestors, and, in particular, the propensity of the patient's ancestors to manifest certain disease phenotypes, or other information pertaining to patterns of genetic inheritance and/or specific genetic mutations.

As before described, genealogic information can be obtained through personal interviews, family histories, and other sources of direct and anecdotal information pertaining to the donor and their ancestors, such as church records, newspapers, and biographies or autobiographies. Genealogic information can also be obtained from existing genealogical databases 54 that have been created by various entities.

For example, the Utah Population Database (UPDB) is a rich source of information for genetic, epidemiological, demographic, and public health studies for residents or former residents of Utah. Researchers have used this resource to identify and study families that have higher than normal incidents of cancer or other diseases, to analyze patterns of genetic inheritance and to identify specific genetic mutations. In addition, demographic studies have shown trends in the fertility transition and changes in mortality patterns for both infants and adults. The central component of the UPDB is an extensive set of Utah family histories, in which family members are linked to demographic and medical information. The UPDB also includes diagnostic records on cancer, cause of death, and medical details associated with births. The UPDB provides access to almost nine million records. These data can only be used for biomedical and health-related research, and the privacy of individuals represented in these records and confidentiality of the data is strictly protected.

The integration of a genealogy criteria component relating to the ancestors of a given patient, in association with routine relevant health-related objective phenotypic attributes for the patient themselves—can create an exponential increase in the value of healthcare monitoring and diagnosis. The capability of identifying a potential disease risk for an individual using a genetic population database, coupled with subsequent monitoring of that patient with subsequent diagnosis, makes it possible to treat the patient in specific, personalized manners using standard procedures and medicines, or using new therapies or medicines. Knowing from a genetic population database that a patient's ancestors possess a gene for a particular trait that is readily identifiable but may be silent through several generations, provides very useful information for the future monitoring and diagnosis of that patient. It makes possible (i) the identification of a potential disease risk for an individual using a genetic population database including genealogy criteria characterizing lineage information pertaining to the individual; and/or (ii) the monitoring the individual for the disease risk; and/or (iii) the treatment of the individual with therapies or medicines that take into account, at least in part, the genealogy criteria.

Various features of the invention are set forth in the following claims.

I/We claim:

1. A system comprising
a biologic material collected from a donor and comprising genetic criteria characterizing a genotype of the donor, phenotype criteria characterizing a phenotype of the donor, and genealogy criteria characterizing lineage information pertaining to the donor,
a unique identifier assigned to the biologic material, and
a biological material record for the biologic material that includes the unique identifier, the genealogy criteria, and at least one of the genetic criteria and the phenotype criteria.

2. A system according to claim 1
wherein the phenotype criteria includes the donor's propensity to manifest a particular disease condition or conditions.

3. A system according to claim 2
wherein the genealogy criteria includes a propensity of an ancestor of the donor to manifest a particular disease phenotype or phenotypes.

4. A system according to claim 1
wherein the genealogy criteria is obtained, at least in part, from a genealogical database.

5. A system according to claim 4
wherein the genealogic database includes the Utah Population Database.

6. A system according to claim 1
wherein the biological material record for the biologic material includes the unique identifier, the genealogy criteria, the genetic criteria, and the phenotype criteria.

7. A system according to claim 1
further including a catalog comprising the biological material record for the biologic material.

8. A system according to claim 7
wherein the catalog comprises a printed medium.

9. A system according to claim 7
wherein the catalog comprises a digital medium.

10. A system according to claim 7
wherein the catalog comprises a relational database.

11. A system according to claim 1
wherein the biologic material is selected from a group consisting of cells, tissue, and a cellular blood product.

12. A system according to claim 1
wherein the biologic material includes human stem and/or progenitor cells.

13. A biologic material sample collected from a donor comprising
a unique identifier assigned to the biologic material sample,
genealogy criteria characterizing lineage information pertaining to the donor,
at least one of a genetic criteria characterizing a genotype of the donor and a phenotype criteria characterizing a phenotype of the donor, and
a biological material record for the biologic material sample that includes the unique identifier, the genealogy criteria, and at least one of the genetic criteria and the phenotype criteria.

14. A sample according to claim 13
wherein the biological material record for the biologic material includes the unique identifier, the genealogy criteria, the genetic criteria, and the phenotype criteria.

15. A sample according to claim 13
wherein the phenotype criteria includes the donor's propensity to manifest a particular disease condition or conditions.

16. A sample according to claim 15
wherein the genealogy criteria includes a propensity of an ancestor of the donor to manifest a particular disease phenotype or phenotypes.

17. A sample according to claim 13
wherein the genealogy criteria is obtained, at least in part, from a genealogical database.

18. A sample according to claim 17
wherein the genealogic database includes the Utah Population Database.

19. A method comprising
collecting a biologic material from a donor,
assigning a unique identifier to the biologic material,
classifying the biologic material according to genealogy criteria characterizing lineage information pertaining to the donor, and at least one of a genetic criteria characterizing a genotype of the donor and a phenotype criteria characterizing a phenotype of the donor, and
creating a biological material record for the biologic material that includes the unique identifier, the genealogy criteria, and at least one of the genetic criteria and the phenotype criteria.

20. A method according to claim 19
further including creating a catalog including the biological material record for the biologic material.

21. A method according to claim 19
wherein classifying the biologic material according to genealogy criteria includes accessing a genealogical database.

22. A method according to claim 21
wherein the genealogic database includes the Utah Population Database.

23. A system comprising
an inventory of biologic materials collected from donors, each biologic material comprising genetic criteria characterizing a genotype of the respective donor, phenotype criteria characterizing a phenotype of the respective donor, and genealogy criteria characterizing lineage information pertaining to the respective donor,
a unique identifier assigned to each one of the biologic materials,
a biological material record for each one of the biologic materials that includes the respective unique identifier, and at least one of the respective genealogy criteria, the respective genetic criteria and the respective phenotype criteria, and
a catalog including the biological material records indexed to identify the biologic materials present in the inventory according to genetic and/or phenotypic and/or genealogic attributes.

24. A system according to claim 23
wherein the catalog comprises a printed medium.

25. A system according to claim 23
wherein the catalog comprises a digital medium.

26. A system according to claim 23
wherein the catalog comprises a relational database.

27. A system according to claim 26
further including a search engine for the relational database program.

28. A system according to claim 23
wherein at least one of the respective genealogy criteria and the respective phenotype criteria manifests a particular disease condition or conditions associated with the donor or an ancestor of the donor.

29. A system according to claim 23
wherein the genealogy criteria is obtained, at least in part, from a genealogical database.

30. A system according to claim 29
wherein the genealogic database includes the Utah Population Database.

31. A biologic material sample collected from a donor comprising a biological material record for the biologic material sample that includes a Biosample Criteria Index Index reflecting a propensity of the donor to manifest a particular selected phenotype and/or genotype, the Biosample Criteria Index being derived from at least one of a genealogy criteria characterizing lineage information pertaining to the donor, a genetic criteria characterizing a genotype of the donor, and a phenotype criteria characterizing a phenotype of the donor.

32. A sample according to claim 31
wherein the biological material record for the biologic material includes a unique identifier, the genealogy criteria, the genetic criteria, and the phenotype criteria.

33. A sample according to claim 32
wherein the phenotype criteria includes the donor's propensity to manifest a particular disease condition or conditions.

34. A sample according to claim 32
wherein the genealogy criteria includes a propensity of an ancestor of the donor to manifest a particular disease phenotype or phenotypes.

35. A sample according to claim 31
wherein the genealogy criteria is obtained, at least in part, from a genealogical database.

36. A sample according to claim 35
wherein the genealogic database includes the Utah Population Database.

37. A method comprising
collecting a biologic material from a donor,
classifying the biologic material according to at least one of a genealogy criteria characterizing lineage information pertaining to the donor, a genetic criteria characterizing a genotype of the donor, and a phenotype criteria characterizing a phenotype of the donor, and
creating a biological material record for the biologic material that includes a Biosample Criteria Index reflecting a propensity of the donor to manifest a particular selected phenotype and/or genotype derived from at least one of the genealogy criteria, the genetic criteria, and the phenotype criteria.

38. A method according to claim 37
further including creating a catalog including the biological material record for the biologic material.

39. A method according to claim 37
wherein classifying the biologic material according to genealogy criteria includes accessing a genealogical database.

40. A method according to claim 39
wherein the genealogic database includes the Utah Population Database.

41. A method comprising
identifying a condition having either congenital and/or acquired origins with resultant acute and/or chronic conditions,
accessing a collection of biologic material obtained from one or more donors, the biologic material being classified, at least in part, according to genealogy criteria characterizing lineage information pertaining to the respective donor, and at least one of a genetic criteria characterizing a genotype of the respective donor and a phenotype criteria characterizing a phenotype of the respective donor,
selecting from the collection one or more biologic materials based at least in part upon the genealogy criteria and/or genetic criteria, and
conducting cell therapy and/or tissue engineering using the selected one or more biologic materials in the treatment and/or research of the condition.

42. A method according to claim 41
wherein collected biologic material is classified, at least in part, according to genealogy criteria obtained by accessing a genealogical database.

43. A method according to claim 42
wherein the genealogic database includes the Utah Population Database.

44. A method according to claim 41
wherein the identified condition includes aging, an autoimmune disease, a blood and/or bleeding disorder, a cardiovascular disease, a cancer, diabetes, a neurology/neurodegenerative disorder, an opthamology disorder, an orthopedic disorder, or renal disease.

45. A method according to claim 41
wherein the phenotype criteria includes the donor's propensity to manifest a particular disease condition or conditions.

46. A method according to claim 41
wherein the genealogy criteria includes a propensity of an ancestor of the individual to manifest a particular disease phenotype or phenotypes.

47. A method comprising
conducting cell therapy and/or tissue engineering using a biologic material classified, at least in part, according to genealogy criteria characterizing lineage information pertaining to a donor of the biologic material, and at least one of a genetic criteria characterizing a genotype of the respective donor and a phenotype criteria characterizing a phenotype of the donor.

48. An assembly comprising
a biological material sample collected from an individual, and
a biological material record uniquely identifying the biological material sample including at least one of a genetic criteria characterizing a genotype of the individual and a phenotype criteria characterizing a phenotype of the individual.

49. An assembly according to claim 48
wherein the biological material record includes genealogy criteria characterizing lineage information pertaining to the individual.

50. An assembly according to claim 48
wherein the biological material record includes a unique identifier assigned to the biological material.

51. An assembly according to claim 48
wherein the biological material record is in written form.

52. An assembly according to claim 48
wherein the biological material record is in digital form.

53. An assembly according to claim 48
wherein the biological material record resides in a digital database.

54. An assembly according to claim 48
wherein the phenotype criteria includes the individual's propensity to manifest a particular disease condition or conditions.

55. An assembly according to claim 48
wherein the genealogy criteria includes a propensity of an ancestor of the individual to manifest a particular disease phenotype or phenotypes.

56. An assembly according to claim 48 wherein the genealogy criteria is obtained, at least in part, from a genealogical database.

57. An assembly according to claim 56
wherein the genealogic database includes the Utah Population Database.

58. A method comprising
identifying a potential disease risk for an individual using a genetic population database including genealogy criteria characterizing lineage information pertaining to the individual, and
monitoring the individual for the disease risk.

59. A method according to claim 58
further including treating the individual with therapies or medicines that take into account, at least in part, the genealogy criteria.

60. A method according to claim 58
wherein the genetic population database includes the Utah Population Database.
